(19) United States
(12) Patent Application Publication
(10) Pub. No.: US 2026/0301188 A1
(43) Pub. Date: Oct. 1, 2026

(54) AUTOMATIC SEGMENTATION OF OCT IMAGES TO PREDICT TREATMENT RESPONSE

(71) Applicants: Genentech, Inc., South San Francisco, CA (US); Hoffmann-La Roche Inc., Little Falls, NJ (US)

(72) Inventors: Andreas MAUNZ, Freiburg (CH); Huanxiang LU, Préverenges (CH); Katie Marie LITTS, South San Francisco, CA (US); Derrek Paul HIBAR, San Francisco, CA (US); Yiyi WANG, South San Francisco, CA (US)

(21) Appl. No.: 19/699,995

(22) Filed: Jun. 5, 2026

Related U.S. Application Data
(63) Continuation of application No. PCT/US24/59222, filed on Dec. 9, 2024.
(60) Provisional application No. 63/608,000, filed on Dec. 8, 2023.
(60) Provisional application No. 63/642,067, filed on May 3, 2024.

(51) Int. Cl. G06T 7/11; G06T 7/00; G16H 15/00; G16H 20/10; G16H 30/40

(52) U.S. Cl. CPC G06T 7/11; G06T 7/0012; G16H 15/00; G16H 20/10; G16H 30/40; G06T 2207/10101; G06T 2207/20081; G06T 2207/20084

(57) ABSTRACT

A method and system for generating a treatment output for a subject with DME includes receiving OCT imaging data for a retina of a subject at a first and a second point in time. Using the OCT imaging data for the first and second point in time, a first and a second OCT segmented image may be generated using a machine learning model. Based on the first and second segmented OCT images, a first and a second measurement of a DME-associated feature may be generated for the first and second point in time. A reduction between the first and second measurements may be identified and compared to a threshold. A reduction that exceeds the threshold may be associated with an improved vision health metric for the subject at a third point in time. Based on the comparison, a treatment output may be generated.

### CROSS-REFERENCE TO RELATED APPLICATIONS

This application is a continuation of International Application No. PCT/US 24/59222 filed Dec. 9, 2024, which claims priority to U.S. Provisional Application No. 63/608,000, filed Dec. 8, 2023, and U.S. Provisional Application No. 63/642,067, filed May 3, 2024, each of which is incorporated herein by reference in its entirety.

### FIELD

The present disclosure relates generally to predicting how subjects with diabetic macular edema will respond to treatment. More particularly, the present disclosure describes methods and systems for automatically segmenting OCT images, using deep learning, to predict treatment response for subjects with diabetic macular edema.

### BACKGROUND

Diabetic Macular Edema (DME) is oftentimes responsible for the vision loss experienced by patients living with diabetes. With DME, excess fluid accumulates in the extracellular space within the retina in the macular area, typically in the inner nuclear layer, outer plexiform layer, Henle's fiber layer, and subretinal space. The current standard of care includes treating patients with DME using a monoclonal antibody treatment, such as Faricimab, or an anti-Vascular Endothelial Growth Factor (anti-VEGF) treatment, such as ranibizumab. However, the treatment response is variable. In particular, different patients may respond differently to the treatment, with many patients not achieving desired outcomes (e.g., certain levels of vision improvement). In some cases, patients may receive more injections or injections at a higher frequency than desired. In other words, the treatment burden for these patients may be higher than desired. Accordingly, there is a need for system or technique that can predict vision outcomes associated with a treatment and therefore reduce treatment burden in patients with DME.

Currently, human analysts may be unable to predict how a given subject will respond to a treatment with a desired level of accuracy, speed, and/or efficiency. For example, human analysts may be unable to predict, with the desired accuracy, speed, and/or efficiency, how an individual subject's vision will improve over a selected number of months after treatment has begun.

### SUMMARY

In one or more embodiments, a method is provided for generating a treatment output for a subject with DME. OCT imaging data for a retina of a subject at a first and a second point in time is received. Using the OCT imaging data for the first and second point in time, a first and a second OCT segmented image may be generated using a machine learning model (e.g., a deep learning model). Based on the first and second segmented OCT images, a first and second measurement of a DME-associated feature may be generated for the first and second point in time. A reduction between the first measurement and the second measurement may be identified. The reduction may be compared to a threshold. A reduction that exceeds the threshold may be associated with an improved vision health metric for the subject at a third point in time. Based on the comparison, a treatment output may be generated.

In one or more embodiments, a system comprises one or more data processors; and a non-transitory computer readable storage medium containing instructions which, when executed on the one or more data processors, cause the one or more data processors to perform one or more of the methods described herein.

In one or more embodiments, a computer-program product tangibly embodied in a non-transitory machine-readable storage medium is provided, which includes instructions configured to cause one or more data processors to perform one or more of the methods described herein.

### BRIEF DESCRIPTION OF THE DRAWINGS

The present disclosure is described in conjunction with the appended figures:

FIG. 1 is a block diagram of a treatment prediction system, in accordance with one or more embodiments.

FIG. 2 is a flowchart diagram of a process for predicting treatment response using the system of FIG. 1, in accordance with one or more embodiments.

FIG. 3 is a block diagram of a retinal segmentation and feature extraction system, in accordance with one or more embodiments.

FIG. 4 is a flowchart diagram of a process for performing retinal segmentation and feature extraction using the system of FIG. 3, in accordance with one or more embodiments.

FIG. 5 is a block diagram that illustrates a computer system, in accordance with one or more embodiments.

It is to be understood that the figures are not necessarily drawn to scale, nor are the objects in the figures necessarily drawn to scale in relationship to one another. The figures are depictions that are intended to bring clarity and understanding to various embodiments of systems and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The embodiments described herein recognize that it may be desirable to have methods and systems for predicting a particular subject's treatment response for a diabetic macular edema (DME) treatment. The treatment may be, for example, a monoclonal antibody treatment (e.g., Faricimab), an anti-Vascular Endothelial Growth Factor (anti-VEGF) treatment (e.g., ranibizumab and/or aflibercept), or another type of treatment. Anti-VEGF treatments primarily target a single pathway to reduce blood vessel leakage and proliferation. DME, however, is a multifactorial disease that can involve other angiogenic factors and inflammatory pathways not addressed with anti-VEGF monotherapy. A more recently developed treatment, Faricimab, which is a bispecific monoclonal antibody, targets both VEGF (e.g., VEGF-A) and the angiopoietin-2 (Ang-2). In particular, Faricimab binds both VEGF-A and Ang-2 with high affinity and specificity.

Currently, human analysts may be unable to predict how a given subject will respond to a treatment with a desired level of accuracy, speed, and/or efficiency. For example, human analysts may be unable to predict, with the desired accuracy, speed, and/or efficiency, how an individual subject's vision will improve over a selected number of months after treatment has begun.

The embodiments described herein recognize that it may be important in a healthcare setting to identify those subjects (patients) who are predicted to have at least a desired response (e.g., at least a certain level of vision improvement) to a treatment. Such predictions may help medical professionals reduce the treatment burden imposed on a subject by a given treatment. In some cases, predicting how a subject will have responded to treatment can help determine whether adjustments need to be made to the dosage or injection frequency of the treatment or whether the treatment needs to be changed.

The embodiments described herein further recognize that it may be important in a clinical trial to identify those subjects who are predicted to have at least a desired response (e.g., at least a certain level of vision improvement) to the treatment(s) being studied as part of that clinical trial. Such predictions may help enrich the population of subjects included in the clinical trial. Similarly, it may be important to identify those subjects who are predicted to not have the desired response to treatment in order to consider excluding such subjects from the clinical trial.

Thus, the embodiments described herein provide methods and systems for predicting the response of a subject to a treatment or clinical trial for DME. The methods and systems described herein automatically segment OCT images, using deep learning (e.g., one or more deep learning models), and using the segmentation, measure one or more retinal layer features and/or retinal pathological elements to determine a predicted treatment response.

In some embodiments, the predicted treatment response may be based on input data. In some embodiments, input data, including image data (e.g., an OCT image), may be input into a segmentation model to generate segmented image data (e.g., a segmented OCT image). In some embodiments, the segmentation model may include a deep learning model that identifies one or more layers of the retina shown in the image data. The segmented image may then be used to measure one or more retinal layer element or retinal pathological elements identified in the segmented image. In various embodiments, one or more volumetric measurements of retinal layer elements and/or retinal pathological elements may be generated using the segmentation system. In some embodiments, the volumetric measurements may be used to analyze the treatment response of a patient for use in the detection, diagnosis, and/or treatment of the patient with DME.

Some of the embodiments described herein may be directed to methods and systems for preparing the data that will be used for predicting the treatment response of the subject. In some embodiments, predicting the treatment response for the subject may be based on image data and/or medical data of the subject. For example, the image data may include an OCT image and the medical data may include a baseline visual acuity (e.g., best corrected visual acuity (BCVA)) measurement, a baseline intraretinal fluid (IRF) volume, a baseline subretinal fluid (SRF) volume, and/or a treatment regime information.

Predicting subject-specific treatment response for a given treatment may help improve overall treatment management of DME. For example, more accurately predicting a specific subject's treatment response may help in the development of more tailored or customized treatment regimens for individual subjects. By predicting whether the treatment protocol will result in a desired response, the healthcare provider may be able to recommend a treatment plan that will result in the best outcome for the subject while minimizing the use of ineffective treatments.

In another example, predicting how the specific subject will respond to a particular treatment may help determine the subjects that should be included in a clinical trial. If the subject is unlikely to respond to the treatment that is being administered to the patient, then it may be advantageous to include them in a clinical trial for a new treatment.

Additionally, using deep learning models to process the input data may reduce the overall computing resources that would be otherwise needed to make such predictions and/or general determinations/recommendations about clinical trials, treatment management, or both. By generating more effective input data, a more efficient and effective segmentation model may also result in saving computing resources.

Recognizing and taking into account the importance and utility of a methodology and system that can provide the improvements described above, the embodiments described in the specification provide methods and systems for improving the accuracy, speed, efficiency, and/or ease of predicting treatment response in subjects with and DME.

### I. Overview of Example Treatment Response Prediction System for DME

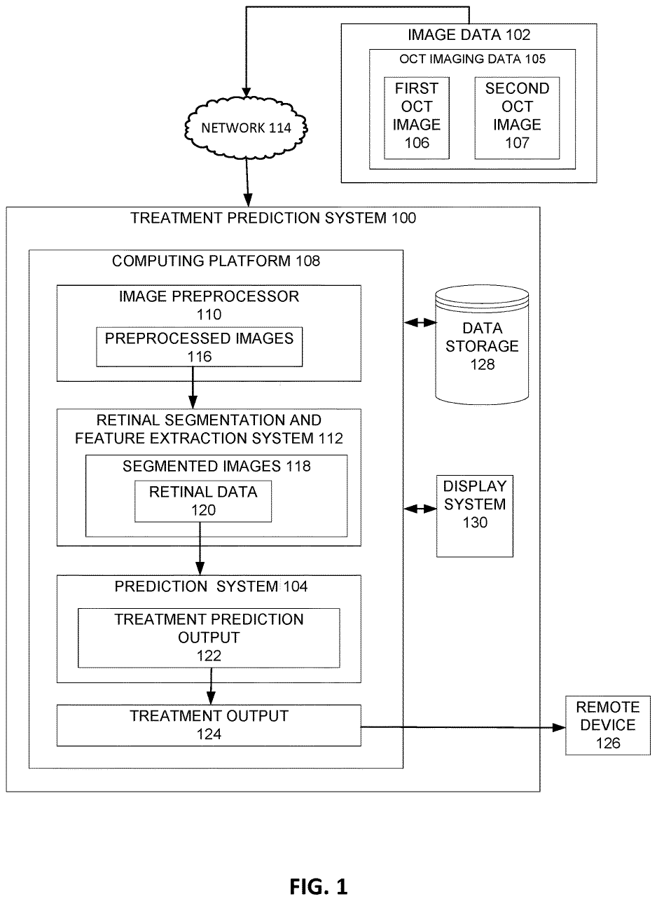

Referring now to the figures, FIG. 1 is a block diagram of a treatment prediction system 100 in accordance with various embodiments. The treatment prediction system 100 uses image data 102 to predict a treatment response for a subject with diabetic macular edema (DME). In particular, the treatment prediction system 100 may include a prediction system 104 that predicts how the eye of a subject with DME will respond to treatment. In some embodiments, the treatment prediction system 100 is used to determine—based on a baseline image and a post-treatment image of the patient (e.g., 4 weeks post-treatment)—a predicted vision health metric at a future point in time (e.g., 1 year post-treatment). In some embodiments, the baseline image and post-treatment image of the patient is used to identify a reduction of IRF volume, and it is determined whether the reduction exceeds a threshold that is associated a predicted vision health metric. In some embodiments, the predicted vision health metric is a prediction that the subject is predicted to have better letter gains (e.g., improved BCVA) at 1 year post-treatment relative to letter gains associated with not exceeding the threshold.

As illustrated, the image data 102 is or includes include OCT imaging data 105. The OCT imaging data 105 may include one or more raw images obtained directly via an imaging system that generated the raw images or the OCT imaging data 105 may include preprocessed raw images. The OCT imaging data 105 may include, for example, without limitation, time domain OCT images (TD-OCT), spectral domain OCT (SD-OCT) images, two-dimensional OCT images (e.g., OCT B scans), three-dimensional OCT images (e.g., OCT volume images), OCT angiography (OCT-A) images, or a combination thereof. An OCT volume may itself be comprised of multiple OCT B-scans. OCT B-scans may include, for example, without limitation, 10s, 100s, 1000s, 10,000s, or some other number of OCT B-scans. An OCT B-scan may also be referred to as an OCT slice image or a cross-sectional OCT image. The OCT imaging data 105 may be generated using an OCT imaging system or OCT scanner. The OCT imaging system can be a large tabletop configuration used in clinical settings, a portable or handheld dedicated system, or a “smart” OCT system incorporated into user personal devices such as smartphones. In some cases, the OCT imaging system may include an image denoiser that is configured to remove noise and other artifacts from a raw OCT volume image to generate an OCT volume. As illustrated, the OCT imaging data 105 includes a first OCT image 106 associated with a subject at a first point in time and a second OCT image 107 associated with the subject at a second point in time that is after the first point in time.

In some embodiments, the image data 102 undergoes a number of processing steps before it is sent to the prediction system 104. As illustrated in FIG. 1, the treatment prediction system 100 includes a computing platform 108 configured to store and execute an image preprocessor 110, a retinal segmentation and feature extraction system 112, and the prediction system 104. While the image preprocessor 110, the retinal segmentation and feature extraction system 112, and the prediction system 104 are illustrated as being stored and executed using the same computing platform (i.e., the computing platform 108), in some embodiments, one or more of the image preprocessor 110, the retinal segmentation and feature extraction system 112, and the prediction system 104 are stored and executed using a computing platform that is different from the computing platform 108. In some embodiments, the retinal segmentation and feature extraction system 112 comprises one or more machine learning models.

Generally, the image preprocessor 110 receives or accesses, for example using a network 114, the image data 102 and performs a set of preprocessing operations on the image data 102 to form preprocessed images 116. The image data 102 may be sent as input into the image preprocessor 110, retrieved by the image preprocessor 110 from storage, or accessed in some other manner. In this example, the image preprocessor 110 is configured or programmed to receive and perform a set of preprocessing operations on the OCT imaging data 105 to form the preprocessed images 116. The set of processing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation. In one or more embodiments, the image preprocessor 110 may be implemented within the computing platform 108 but in other embodiments at least a portion of (e.g., a module of) the image preprocessor 110 is implemented within an imaging system, which may be or include an OCT imaging system. In some embodiments, the preprocessed image(s) 116 are sent to the retinal segmentation and feature extraction system 112. In some embodiments, the preprocessed images 116 include a first preprocessed image based on the first OCT image 106 and a second preprocessed image based on the second OCT image 107.

In some embodiments, the preprocessed images 116 require additional processing before being sent to the prediction system 104. As such, the preprocessed images 116 may be sent to the retinal segmentation and feature extraction system 112, which generates segmented images 118 using the preprocessed images 116. The preprocessed images 116 may be sent as input into the retinal segmentation and feature extraction system 112, retrieved by the retinal segmentation and feature extraction system 112 from storage, or accessed in some other manner. In some embodiments, the retinal segmentation and feature extraction system 112 identifies retinal features on the segmented images and may generate retinal data 120 based on the identified retinal features. The identified retinal features and related retinal data 120 may be included in the segmented images 118 and may be used by the prediction system 104. As such, the segmented images may be image input for the prediction system 104.

In some embodiments, one or more of the segmented images may be generated from the preprocessed images 116 and/or the image data 102 according to one or more techniques as described in International Publication No. WO2023205511A1 and International Publication No. WO2023250417A1, each of which is incorporated by reference herein in its entirety. Moreover and in some embodiments, the retinal segmentation and feature extraction system 112 is or includes one or more of the systems for automated retinal segmentation as described in International Publication No. WO2023205511A1 and/or the ophthalmic analysis and measurement system as described in International Publication No. WO2023250417A1.

The segmented images 118 may include the retinal data 120. The retinal data 120 may include values for various retinal features relating to one or more pathological elements of the retina, one or more layers of the retina, or both. The retinal data 120 may include, for example, without limitation, feature data extracted from segmented image data generated by the retinal segmentation and feature extraction system 112. For example, feature data may be extracted for one or more retinal elements identified in the segmented image data. This feature data may include values for any number of or combination of features (e.g., quantitative features). These features may include pathology-related features, layer-related volume features, layer-related thickness features, or a combination thereof. Examples of features include, but are not limited to, a volume of a retinal fluid pocket, a maximum retinal layer thickness, a minimum retinal layer thickness, an average retinal layer thickness, a maximum height of a boundary associated with a retinal layer, a length of a fluid pocket, a width of a fluid pocket, a number of retinal fluid pockets, and a number of hyperreflective foci. Thus, at least some of the features may be volumetric features. For example, the retinal data 120 may be derived for each selected OCT image (e.g., single OCT B scan) and then combined to form volume-wide values. In some embodiments, the retinal data 120 may be derived for each selected OCT image and then aggregated over the Early Treatment Diabetic Retinopathy Study (“ETDRS”) grid. In some embodiments, the retinal data 120 includes data relating to a DME-associated feature such as for example IRF fluid. In some embodiments, the data relating to the DME-associated feature is a measurement, such as for example IRF volume. In some embodiments, the segmented images 118 include a first segmented image based on the first preprocessed image associated with the subject at the first point in time and a second segmented image based on the second preprocessed image associated with the subject at the second point in time. In some embodiments, the segmented images include data relating to a DME-associated feature that is present in both the first segmented image and the second segmented image. Additional detail is provided below in Section III for one example of the retinal segmentation and feature extraction system 112.

In some embodiments, the prediction system 104 uses the segmented images 118, which includes the retinal data 120, from the retinal segmentation and feature extraction system 112 to generate a treatment prediction output 122, which predicts the treatment response for a subject with DME. In some embodiments, and when the prediction system 104 receives a first segmented image associated with the first point in time and the second segmented image associated with the second point in time, a change to a DME-associated feature can be determined. In some embodiments, the change is a reduction in IRF volume between the first OCT image 106 and the second OCT image 107. In some embodiments, the treatment prediction output 122 includes an identification of the subject associated with the image data 102 as a subject with a change that exceeds or does not exceed a threshold. In some embodiments, the threshold may be a threshold related to a reduction in a DME-related measurement. In some embodiments, the predicted treatment response includes a predicted vision health metric, which may be an indication that the subject is predicted to have better letter gains at 1 year post-treatment. In some embodiments, better letter gains at 1 year post-treatment is relative to letter gains at 1 year post-treatment when the reduction does not meet the threshold. In one or more embodiments, the treatment prediction output 122 may be defined using one or more different types of vision-related outcomes, such as for example vision acuity, a predicted macular thickness, one or more other types of predicted visual outcome metrics, or a combination thereof. In some examples, the treatment prediction output 122 may be a predicted measurement at a future point in time, such as for example a predicted best corrected visual acuity (BCVA) at the future point in time (e.g., BCVA predicted at 1 year post-treatment); a predicted central subfield thickness (CST) at the future point in time (e.g., CST predicted at week 24 post-treatment); and/or a predicted difference between the CST at a first point in time, such as at baseline, and a second point in time, such as 6 weeks, 18 weeks, 24 weeks, 1 month, 3 months, 4 months, 5 months, 6 months, 1 year, 2 years or some other amount of time after treatment has begun. In some embodiments, the treatment prediction output 122 is a categorial outcome prediction. The prediction system 104 may be implemented using hardware, software, firmware, or a combination thereof.

In some embodiments, the treatment prediction output 122 is used to generate a treatment output 124. In some embodiments, the treatment output 124 may include a treatment recommendation for the subject based on the treatment prediction output 122. In some embodiments, and when the subject is identified as having a reduction that exceeds the threshold and with respect to a first treatment regimen, then the treatment output 124 may include a recommendation to continue with the first treatment regimen. That is, when exceeding the threshold is associated with a desired response to the first treatment regimen and the reduction associated with the subject exceeds the threshold, then the treatment output 124 includes a recommendation to continue with the first treatment regimen. However, in other embodiments and when the subject is identified as having a reduction that does not exceed a threshold, then the treatment output 124 may include a recommendation to change from the first treatment regimen. That is, when the subject is predicted by the prediction system 104 to have a less desirable response to the first treatment regimen, then the treatment output 124 may include administering a treatment regime that is different from the first treatment regimen.

In some embodiments, when the subject is identified as having a reduction in a measurement—in response to a monoclonal antibody treatment, such as Faricimab—that exceeds the threshold, then the treatment output 124 may include a recommendation to continue administering the monoclonal antibody treatment, such as Faricimab. In some embodiments, when the subject is identified as having a reduction in measurement—in response to an anti-Vascular Endothelial Growth Factor (anti-VEGF) treatment, such as ranibizumab—that exceeds the threshold, then the treatment output 124 may include a recommendation to continue administering the anti-Vascular Endothelial Growth Factor (anti-VEGF) treatment, such as ranibizumab. In some embodiments, the treatment output 124 may include, for example, the treatment prediction output 122.

In some embodiments, the treatment output 124 includes other types of information. For example, in some cases, the treatment output 124 includes a clinical trial recommendation, the treatment recommendation, or both. A clinical trial recommendation may be a recommendation to include or exclude the subject from a clinical trial. The treatment recommendation may be a recommendation to change the type of treatment that will be given to the subject, adjust the treatment regimen (e.g., injection frequency, dosage, etc.) for the treatment, consider a new type of treatment, add a new treatment to the existing treatment regimen, or alter the treatment plan for the subject in some other manner.

In some embodiments, the treatment output 124 is sent to a remote device 126 via the network 114. The treatment prediction system 100 also includes a data storage 128 and a display system 130. The data storage 128 and the display system 130 are each in communication with the computing platform 108. In some examples, the data storage 128, the display system 130, or both may be considered part of or otherwise integrated with the computing platform 108. Thus, in some examples, the computing platform 108, the data storage 128, and the display system 130 may be separate components in communication with each other, but in other examples, some combination of these components may be integrated together. In one or more embodiments, the computing platform 108 includes a single computer (or computer system) or multiple computers in communication with each other. In other examples, the computing platform 108 takes the form of a cloud computing platform, a mobile computing platform (e.g., a smartphone, a tablet, etc.), or a combination thereof.

At least a portion of treatment output 124 or a graphical representation of at least a portion of treatment output 124 may be displayed on the display system 130. In some embodiments, at least a portion of treatment output 124 or a graphical representation of at least a portion of treatment output 124 is sent to remote device 126 (e.g., a mobile device, a laptop, a server, a cloud, etc.).

Predicting subject-specific treatment response using the treatment prediction system 100 for a given treatment is more accurate and may help improve overall treatment management of DME. For example, more accurately predicting a specific subject's treatment response using the treatment prediction system 100 may help in the development of more tailored or customized treatment regimens for individual subjects. By predicting, using the treatment prediction system 100, whether the treatment protocol will result in a desired response, then a healthcare provider may be able to recommend a treatment plan that will result in the best outcome for the subject while minimizing the use of ineffective treatments.

In another example, predicting how the specific subject will respond to a particular treatment using the treatment prediction system 100 may help determine the subjects that should be included in a clinical trial. If the subject is not predicted to have a desirable response to his or her existing treatment regime, then it may be advantageous to include them in the clinical trial involving a new treatment regime.

Additionally, using the image preprocessor 110 and/or the retinal segmentation and feature extraction system 112 to process the input data may reduce the overall computing resources that would be otherwise needed to make such predictions and/or general determinations/recommendations about clinical trials, treatment management, or both.

In some embodiments, the treatment prediction system 100 predicts treatment response for patients with better accuracy and consistency than expert human graders. The treatment prediction system 100 provides a technical effect of improving accuracy, reducing the overall computing resources, and/or reducing the time needed to predict a specific subject's predicted treatment response. Further, using the treatment prediction system 100 may help improve overall treatment management of DME as compared to other methods and systems.

In some embodiments, the treatment prediction system 100 provides a technical improvement to the field of DME treatment and/or the technical field of predicting a specific subject's predicted treatment response. As noted above, the treatment prediction system 100 predicts treatment response for patients with better accuracy and consistency than expert human graders and may reduce the overall computing resources and/or time needed to predict treatment response for subjects.

### II. Example Methodologies for Predicting Treatment Response for DME

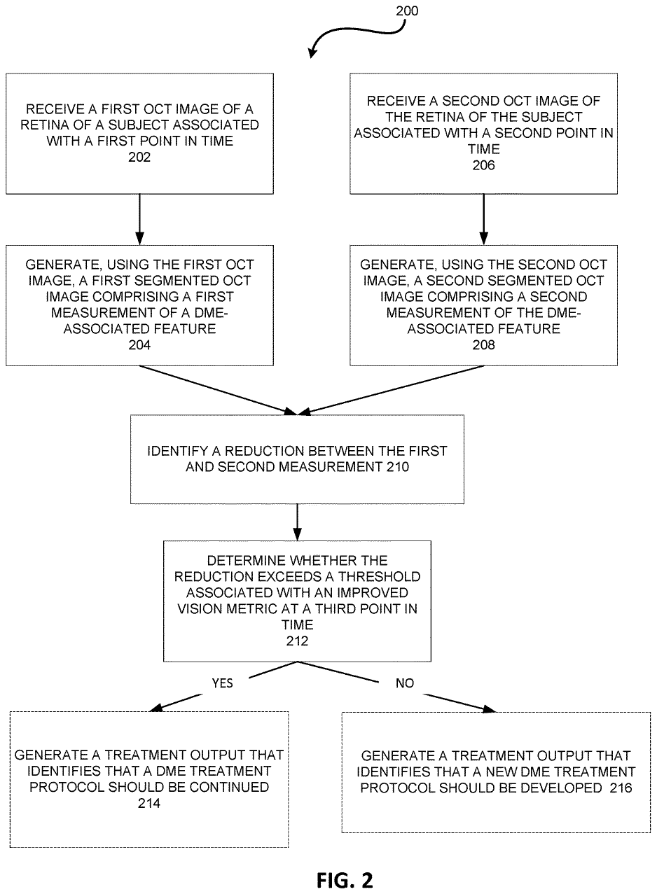

FIG. 2 is a flowchart of a process 200 for predicting treatment response in accordance with one or more embodiments. In one or more embodiments, the process 200 may be implemented using the treatment prediction system 100 described in FIG. 1. The process 200 includes various steps and may be described with continuing reference to FIG. 1. One or more steps that are not expressly illustrated in FIG. 2 may be included before, after, in between, or as part of the steps of the process 200. In some embodiments, the process 200 may begin with step 202.

As illustrated in FIG. 2, the process 200 includes, at step 202, receiving a first OCT image of a retina of a subject associated with a first point in time. In various embodiments, the first OCT image may be the first OCT image 106 in FIG. 1. In some embodiments, the first OCT image is a baseline image. In one or more embodiments, the baseline point in time may be a point in time prior to treatment, the same day as a treatment dose (e.g., a first treatment dose), the same day as an initial diagnosis of DME, the same day as a screening conducted for DME, or some other type of baseline or reference point in time. The OCT image may be, for example, an SD-OCT image or a TD-OCT image.

The process 200 further includes, at step 204, generating a first segmented OCT image using a segmentation and feature extraction system and the first OCT image. In some embodiments, the first segmented OCT image is one of the segmented images 118 in FIG. 1. In some embodiments, the first segmented OCT image comprises a first measurement of a DME-associated feature. In one embodiment, the DME-associated feature is intraretinal fluid volume (IRF) of the retina of the subject with DME at the baseline point in time. The segmentation and feature extraction system may be, for example, the segmentation and feature extraction system 112 in FIG. 1. In some embodiments, the first segmented OCT image includes the first measurement of the DME-associated feature and is generated by the segmentation and feature extraction system 112. In some embodiments, the first measurement of the DME-associated feature is a baseline measurement of IRF volume.

The process 200 further includes, at step 206, receiving a second OCT image of the retina of the subject associated with a second point in time. In various embodiments, the second OCT image may be the second OCT image 107 in FIG. 1. In some embodiments, the second OCT image is between about 2 weeks and about 6 weeks post-treatment. In some embodiments, the second OCT image is at or at about 4 weeks post-treatment. The OCT image may be, for example, an SD-OCT image or a TD-OCT image. In one or more embodiments, the second point in time may be 4 weeks after a subject begins a treatment regime or 4 weeks after a patient receives a treatment dose. In one or more embodiments, the treatment may include treatment with Faricimab. In other embodiments, the treatment may include treatment with aflibercept. In one or more embodiments, the treatment protocol may include the type of medication, the medication dosage frequency, including number of injections and time period between injections, and/or other particulars of a patient's treatment regime.

The process 200 further includes, at step 208, generating a second segmented OCT image using a segmentation and feature extraction system and the second OCT image. In some embodiments, the second segmented OCT image is one of the segmented images 118 in FIG. 1. In some embodiments, the second segmented OCT image comprises a second measurement of the DME-associated feature that was measured in the first segmented OCT image. In one embodiment, the DME-associated feature is intraretinal fluid volume (IRF) of the retina of the subject with DME at the second point in time. The segmentation and feature extraction system may be, for example, the segmentation and feature extraction system 112 in FIG. 1. In some embodiments, the second segmented OCT image includes the second measurement of the DME-associated feature and is generated by the segmentation and feature extraction system 112. In some embodiments, the second measurement of the DME-associated feature is a 4 week post-treatment measurement of IRF volume.

The process 200, further includes, at step 210, identifying a reduction between the first and second measurements. In some embodiments, the reduction is a reduction in IRF volume. The step 210 is conducted, for example, by measuring the difference between the baseline IRF volumetric measurement and the week 4 IRF volumetric measurement. In various embodiments, the change may be a numerical value. In various embodiments, the change may be a percentage. In various embodiments, the step 210 may be implemented by a computing platform. For example, the step 210 may be implemented by the computing platform 108 in FIG. 1. In other embodiments, step 210 may be conducted by human analysts.

The process 200 further includes, at step 212, determining whether the reduction exceeds a threshold associated with improved vision metric at a third point in time. In some embodiments, the third point in time a future point in time such as or example 4 weeks, 1 month, 3 months, 6 months, 9 months, 10 month, 11 months after the second point in time. In some embodiments, the second OCT image 107 is the most recent OCT image associated with the subject and the third point in time is a future point in time (e.g., 1 year post-treatment). In some embodiments, the threshold is, or is about, 50% reduction. In other embodiments, the threshold is, or is about, 40%, 45%, 55%, 60%, or 65%. In some embodiments, the threshold is dependent upon the DME-associated feature being measured. As such, and for a DME-associated feature that is not IRF volume, the threshold may be less than 50%, such as for example 40%, 30%, or 20%. The threshold may be dependent upon the period of time between the first time period and the second time period. As such, while at week 4 post-treatment, the threshold is 50% in one example, if the reduction is associated with a two week period of time (as opposed to a 4 week period of time), the threshold may be less than 50% such as for example 40%, 30%, or 20%. In some embodiments, and at the step 212, it is determined whether the reduction in IRF volume between the baseline measurement and the second point of time (e.g., 4 week post-treatment) exceeds the 50% threshold. In various embodiments, the step 412 may be determined by a computing platform. For example, the step 212 may be implemented by computing platform 108 in FIG. 1. In other embodiments, step 212 may be determined by human analysts.

The process 200 optionally includes, at step 214, generating a treatment output based on the subject exceeding the threshold. In some embodiments, the treatment output is the treatment output 124 in FIG. 1. In some embodiments, the treatment output identifies that a DME treatment protocol should be continued for a subject when the change in IRF volume is greater than the 50% threshold. As discussed below in Section V, patients with greater than 50% IRF volume reduction between the baseline measurement and the week 4 measurement had greater improvements in BCVA scores at 1 year post-treatment. A subject exceeding the 50% threshold may suggest, for example, that the current medication, treatment regime, and/or clinical trial the patient is currently on/participating in is effective in treating, or at least maintaining, DME progression and symptoms in the patient. Thus, an appropriate treatment for those patients identified as exceeding the 50% threshold may be continuing their existing treatment regime.

The process 200 optionally includes, at step 216, generating a treatment output based on the subject not exceeding the threshold. In some embodiments, the treatment output is the treatment output 124 in FIG. 1. In some embodiments, the treatment output identifies that a new DME treatment protocol should be developed for the subject when the reduction in IRF volume between the baseline measurement and the week 4 measurement is less than the 50% threshold. As noted above, if the subject with DME experiences less than 50% reduction in IRF volume within 4 weeks of starting treatment, it may be an indication that the current medication, treatment regime, or clinical trial is not as effective at treating that patient's DME symptoms or progression. Thus, it may be decided that a different treatment protocol should be developed for the patient. This could include, for example, a different medication, a different injection frequency, exclusion from a current clinical trial, or admission into a different clinical trial.

### III. Example Retinal Segmentation and Feature Extraction System

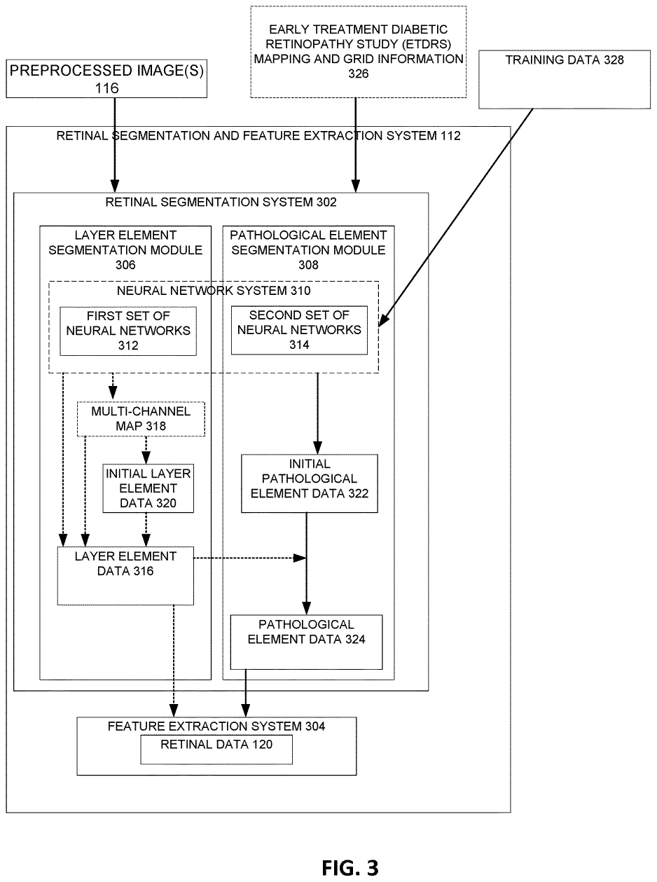

FIG. 3 is a block diagram of the retinal segmentation and feature extraction system 112. Generally, the retinal segmentation and feature extraction system 112 generates segmented images 118 and the retina data 120 in FIG. 1. In some embodiments, the retinal segmentation and feature extraction system 112 includes a retinal segmentation system 302 and a feature extraction system 304, which uses output of the retinal segmentation system 302 to generate the retinal data 120.

In some embodiments, the retinal segmentation system 302 includes a layer element segmentation module 306 and a pathological element segmentation module 308, each of which may be implemented using software, firmware, hardware, or a combination thereof. In one or more embodiments, the layer element segmentation module 306 and the pathological element segmentation module 308 are separate modules that work together to perform automated retinal segmentation. In other embodiments, the layer element segmentation module 306 and the pathological element segmentation module 308 may be integrated together within a single module. The layer element segmentation module 306 and the pathological element segmentation module 308 are used in two different pathways of processing.

The layer element segmentation module 306 is used to perform layer element segmentation to detect and identify retinal layer elements. A retinal layer element may be, for example, a retinal layer or a boundary associated with a retinal layer. Examples of retinal layers include, but are not limited to, an internal limiting membrane (ILM) layer, an external limiting membrane (ELM) layer, an outer plexiform layer-Henle fiber layer (OPL-HFL), a retinal pigment epithelial (RPE) layer, a layer of RPE detachment, a Bruch's membrane (BM) layer, an ellipsoid zone (EZ), and other types of retinal layers. A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of an RPE (IB-RPE) detachment layer, an outer boundary of the RPE (OB-RPE) detachment layer, or another type of boundary.

The pathological element segmentation module 308 is used to perform pathological element segmentation to detect and identify retinal pathological elements. A retinal pathological element may include, for example, fluid, cells, solid material, or a combination thereof that evidences a retinal pathology associated with an ophthalmological disease or condition. For example, the presence of certain retinal fluids, like intraretinal fluid, may be a sign of DME. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, and a disruption. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption.

Additionally, a retinal pathological element may include a characteristic or subtype of one of the fluids (e.g., IRF, SRF, fluid associated with PED), materials (e.g., HRM, SHRM, IHRM), lesions (e.g., HRF, SHRM lesions), or disruptions. In particular, examples of retinal pathological elements may include characteristics and/or subtypes of the different types of elements and disruptions described above that can be detected and identified via retinal segmentation. For example, whether a retinal fluid is clear or turbid may be detectable and identifiable characteristic of the retinal fluid. Accordingly, in some examples, a retinal pathological element may be clear IRF, turbid IRF, clear SRF, turbid SRF, some other type of clear retinal fluid, some other type of turbid retinal fluid, or a combination thereof. In some cases, for SHRM, shape characteristics (e.g., tall SHRM, dome-shaped SHRM at the foveal center, flat SHRM near the foveal center, dysmorphic, etc.), boundary characteristics (e.g., ill-defined SHRM, well-defined SHRM), reflectivity (e.g., increased reflectivity or other levels of reflectivity), layering characteristics (e.g., hyperreflective bands in SHRM lesions), and lesion characteristics (e.g., the height, width, and/or area of SHRM lesions) may be examples of retinal pathological elements that may be detected and identified via retinal segmentation.

In some cases, a retinal layer element is associated with a retinal pathological element. For example, an RPE detachment layer, which is a retinal layer element, is associated with PED, which is a retinal pathological element. Accordingly, the layer element segmentation module 306 and the pathological element segmentation module 308 may communicate with each other in order to automatically and more accurately perform retinal segmentation.

In one or more embodiments, the retinal segmentation system 302 uses a machine learning system to perform the automated segmentation. In one or more embodiments, a neural network system 310 comprises a deep learning model. The neural network system 310 may include any number of or combination of neural networks. In one or more embodiments, the neural network system 310 takes the form of a convolutional neural network (CNN) system that includes one or more convolutional neural networks. For example, the CNN may include a plurality of neural networks, each of which may itself be a convolutional neural network.

In one or more embodiments, a first portion of neural network system 310 is implemented within the layer element segmentation module 306, while a second portion of the neural network system 310 is implemented within the pathological element segmentation module 308. For example, the layer element segmentation module 306 may include a first set of neural networks 312 of the neural network system 310; the pathological element segmentation module 308 may include a second set of neural networks 314 of the neural network system 310.

Each of the first set of neural networks 312 and the second set of neural networks 314 may be itself comprised of a set of neural networks. In one or more embodiments, the first set of neural networks 312 and the second set of neural networks 314 differ by at least one neural network. In other words, the second set of neural networks 314 may include at least one neural network that is different from the one or more neural networks in the first set of neural networks 312. In other embodiments, the first set of neural networks 312 and the second set of neural networks 314 may include the same one or more types of neural networks. For example, the same one or more types of neural networks may be used to perform both layer element segmentation and pathological element segmentation. In some cases, the first set of neural networks 312, the second set of neural networks 314, or both may include one or more mathematical algorithms or functions in addition to a set of neural networks.

In one or more embodiments, the image data 102 and/or the preprocessed image 116 is processed along a first pathway using the layer element segmentation module 306, which uses the first set of neural networks 312 to perform automated layer element segmentation. For example, the layer element segmentation module 306 may receive the preprocessed image 116 and/or the image data 102 at the first set of neural networks 312 for processing. In some embodiments, the layer element segmentation module 306 preprocesses the image data 102 and/or the preprocessed image 116 to enable focused attention on particular regions of interest prior to inputting the image data 102 and/or the preprocessed image 116 into the first set of neural networks 312. This preprocessing may include, for example, reducing noise and/or artifacts in the image data 102 and/or the preprocessed image 116 that might otherwise impair the ability to properly assess particular regions of interest. In some cases, the first set of neural networks 312 is trained to preprocess the image data 102 and/or the preprocessed image 116.

The layer element segmentation module 306 uses the first set of neural networks 312 to process the input received (e.g., the image data 102 and/or the preprocessed image 116) to perform automated layer element segmentation and the generate layer element data 316 for a set of retinal layer elements detected within the image data 102 and/or the preprocessed image 116. The layer element data 316 may include, for example, without limitation, a layer element image (which may also be referred to as a layer element segmented image), pixel data that assigns each pixel or section of pixels to a retinal layer element, image coordinates that map out each retinal layer element, other information for the set of retinal layer elements that have been detected, or a combination thereof.

A layer element image, which may be a layer element OCT image, includes a set of graphical indicators, which may be referred to as a set of layer element indicators. The set of layer element indicators identifies a set of retinal layer elements. A layer element indicator may take the form of, for example, without limitation, a color indicator, a shape indicator, a pattern indicator, a shading indicator, a line, a curve, a marker, a label, a tag, text, another type of graphical indicator, or a combination thereof. In some cases, two or more layer element indicators may identify a same retinal layer element. For example, a particular color may be used to identify pixels that represent a particular retinal layer element, while a label may be used to name or identify the particular retinal layer element associated with the particular color.

In some examples, a layer element indicator for identifying a retinal layer element that is a boundary associated with a retinal layer takes the form of a colored and/or patterned curve (continuous or discontinuous) on the layer element image. This curve represents the boundary. In other examples, a layer element indicator for identifying a retinal layer element that is a retinal layer may take the form of a colored and/or patterned region or shape (continuous or discontinuous) on the layer element image. The region or shape may represent, for example, the full thickness of the corresponding retinal layer.

In some embodiments, the first set of neural networks 312 receives the image data 102 and/or the preprocessed image 116 and generates a multi-channel map 318, which is then used to generate the layer element data 316. The multi-channel map 318 may be comprised of a plurality of segmented images, with each segmented image of the plurality of segmented images corresponding to a different retinal layer element or a different retinal layer of interest. As one example, the plurality of segmented images may include a different segmented image for each retinal layer element of interest. As another example, the plurality of segmented images may include a different segmented image for each retinal layer of interest. In some cases, there may be two or more retinal layer elements of interest corresponding to a same retinal layer (e.g., an inner boundary and an outer boundary for the same retinal layer).

The first set of neural networks 312 may output the multi-channel map 318 and the layer segmentation module 306 may further process the multi-channel map 318 using any number of or combination of various mathematical techniques (e.g., curve approximation, logistic function(s), smoothing function(s), another type of function or algorithm, or a combination thereof) to generate the layer element data 316. In other embodiments, the multi-channel map 318 may be produced as an intermediate output by the first set of neural networks 312, which then uses the multi-channel map 318 to generate the layer element data 316 as the output of the first set of neural networks 312.

In still other embodiments, the multi-channel map 318 may be processed to generate the initial layer element data 320 that is then refined to form the layer element data 316 (which can then be referred to as refined layer element data). The initial layer element data 320 may include, but is not limited to, a layer element image (which may also be referred to as a layer element segmented image), pixel data that assigns each pixel or section of pixels to a retinal layer element, image coordinates that map out each retinal layer element, other information of the set of retinal layer elements that have been detected, or a combination thereof. But in these examples, the initial layer element data 320 may be a first approximation.

As one example, the initial layer element data 320 may include an initial layer element image having at least one layer element indicator that identifies a boundary associated with a retinal layer of interest. This initial layer element image may be processed using any number of or combination of various mathematical techniques (e.g., curve approximation, smoothing function(s), another type of function or algorithm, or a combination thereof) to refine the initial layer element image and generate a refined layer element image that forms at least a portion of the layer element data 316. In one or more embodiments, this refinement may be a smoothing of the identified boundary.

In other embodiments, the initial layer element data 320 may be produced as an intermediate output by the first set of neural networks 312, which then uses the initial layer element data 320 to generate the layer element data 316 as the output of the first set of neural networks 312. In this manner, the layer element data 316 may be generated in any number of different ways by the layer element segmentation module 306 within the first pathway of processing.

In one or more embodiments, the image data 102 and/or the preprocessed image 116 is also processed along a second pathway using the pathological element segmentation module 308, which uses the second set of neural networks 314 of the neural network system 310 to perform pathological element segmentation. For example, the pathological element segmentation module 308 may receive the image data 102 and/or the preprocessed image 116 at the second set of neural networks 314 for processing. In some embodiments, the pathological element segmentation module 308 preprocesses the image data 102 and/or the preprocessed image 116 to enable focused attention on particular regions of interest prior to inputting the image data 102 and/or the preprocessed image 116 into the second set of neural networks 314. This preprocessing may include, for example, reducing noise and/or artifacts in the image data 102 and/or the preprocessed image 116 that might otherwise impair the ability to properly assess particular regions of interest. In some cases, the second set of neural networks 314 is trained to the preprocess the image data 102 and/or the preprocessed image 116.

The pathological element segmentation module 308 uses the second set of neural networks 314 to process the input received (e.g., the image data 102 and/or the preprocessed image 116) to perform automated pathological element segmentation and generate the initial pathological element data 322 for a set of pathological layer elements detected within the image data 102 and/or the preprocessed image 116. The initial pathological element data 322 may include, for example, without limitation, a pathological element image (which may also be referred to as a pathological element segmented image), pixel data that assigns each pixel or section of pixels to a retinal pathological element, image coordinates that map out each retinal pathological element, other information for the set of retinal pathological elements that have been detected, or a combination thereof.

A pathological element image, which may be a pathological element OCT image, includes a set of graphical indicators, which may be referred to as a set of pathological element indicators. The set of pathological element indicators identifies a set of retinal pathological elements. A pathological element indicator may take the form of, for example, without limitation, a color indicator, a shape indicator, a pattern indicator, a shading indicator, a line, a curve, a marker, a label, a tag, text, another type of graphical indicator, or a combination thereof. In some cases, two or more pathological element indicators may identify a same retinal pathological element. For example, a particular color may be used to identify pixels that represent a particular retinal pathological element, while a label may be used to name or identify the particular retinal pathological element associated with the particular color.

In some examples, a pathological element indicator for identifying a retinal pathological element that is a retinal fluid may take the form of a colored and/or patterned region or shape (continuous or discontinuous) on the pathological element image. The region or shape may represent, for example, the pocket formed by the retinal fluid.

The initial pathological element data 322 output from the second set of neural networks 314 may then be further processed and refined by the pathological element segmentation module 308. For example, the pathological element segmentation module 308 receives layer element data 316 (or at least a portion of the layer element data 316) from the layer element segmentation module 306. The pathological element segmentation module 308 uses both the initial pathological element data 322 and the layer element data 316 to refine the initial pathological element data 322 and generate the pathological element data 324, which may be referred to as refined pathological element data.

Similar to the initial pathological element data 322, the pathological element data 324 may include, for example, without limitation, a pathological element image (which may also be referred to as a pathological element segmented image), pixel data that assigns each pixel or section of pixels to a retinal pathological element, image coordinates that map out each retinal pathological element, other information for the set of retinal pathological elements that have been detected, or a combination thereof. The pathological element data 324 more accurately identifies and locates the set of retinal pathological elements that are of interest as compared to the initial pathological element data 322. For example, the pathological element data 324 includes a refined pathological element image with a set of pathological element indicators, this set of pathological element indicators may more accurately identify at least one corresponding retinal pathological element as compared to initial pathological element data 322.

In one or more embodiments, the pathological element segmentation module 308 uses the layer element data 316 to constrain the allowable area for the set of retinal pathological elements identified in the pathological element data 324. For example, a portion of the layer element data 316 corresponding to two retinal layers may be used to constrain the allowable area for a retinal pathological element such that the retinal pathological element is not identified as extending beyond the allowable area for the retinal pathological element. As one specific example, the layer element data 316 may be used to constrain the allowable area for an intraretinal fluid in the pathological element image such that the intraretinal fluid is not identified by a corresponding pathological element indicator as crossing over into a subretinal space.

Thus, the layer element data 316 can be used to refine the anatomic characterization of a retinal pathological element of the set of retinal pathological elements identified in the pathological element data 324 using the one or more pathological element indicators that correspond to the retinal pathological element. The anatomic characterization of a retinal pathological element may include at least one of, for example, without limitation, the location, size, shape, length, width, thickness, volume, or other characteristic of the retinal pathological element.

In other embodiments, the initial pathological element data 322 may be an intermediate output of the second set of neural networks 314 and the layer element data 316 may be input into the second set of neural networks 314 to refine the initial pathological element data 322. In these examples, the second set of neural networks 314 outputs the pathological element data 324.

Refining the initial pathological element data 322 using the layer element data 316 improves the overall accuracy of the pathological element segmentation module 308 generating the pathological element data 324. This improvement in accuracy may be carried through in any future analysis conducted using the pathological element data 324.

For example, the feature extraction system 304 may be implemented in the computing platform 108. The feature extraction system 304 may be used to automatically extract the retinal data 120 from the pathological element data 324 and, in some cases, the layer element data 316. The retinal data 120 may include values for any number of or combination of features (e.g., quantitative features). Examples of such features may include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, and a number of hyperreflective foci.

Refining the initial pathological element data 322 to form (refined) the pathological element data 324 improves the accuracy of the retinal data 120 that is extracted. Further, any detection, diagnosis, and/or treatment methodologies that rely on the pathological element data 324 and/or the retinal data 120 extracted from the pathological element data 324 may be more accurate.

In one or more embodiments, the retinal data 120 includes values for features that are associated with the ETDRS grid. In some embodiments, the retinal segmentation and feature extraction system 112 accesses ETDRS mapping and grid information 326.

In one or more embodiments, the OCT imaging data 105 may be associated with the ETDRS mapping and grid information 326. For example, OCT imaging data 105 may be generated along with one or more 2D images (e.g., en face image, infrared image, thickness map) each corresponding, for example, to the anatomic center of the patient's macula. In one or more embodiments, the ETDRS grid may be constructed to correspond to a 2D image of a patient's macula and may be superimposed over the one or more 2D images (e.g., en face image, infrared image, thickness map). In one or more embodiments, the ETDRS mapping information may identify the location of one or more features of interest or areas of interest in terms of the nine subfields of the ETDRS grid, including, for example: “Center” =Center point of macula; Inner ring: ISS=“Inner Superior Subfield”; INS=“Inner Nasal Subfield”; “Inner Interior Subfield”; IIS=“Inner Inferior Subfield”; ITS=“Inner Temporal Subfield”; Outer ring: OSS=“Outer Superior Subfield”; ONS=“Outer Nasal Subfield”; OIS=“Outer Inferior Subfield”; OTS=“Outer Temporal Subfield.

In one or more embodiments, the ETDRS mapping and grid information 326 may be utilized to determine one or more volumetric measurements (e.g., volume, thickness, area, extent, area of disruption, presence or absence of one or more disease-associated features, a number of disease-associated features) for qualifying and quantifying diabetic macular edema (DME) from the image data 102 and/or the preprocessed image 116. For example, the image data 102 and/or the preprocessed image 116 may be generated along with one or more 2D images (e.g., en face image, infrared image, thickness map) each corresponding, for example, to the anatomic center of the patient's macula.

In one or more embodiments, the ETDRS grid may bounded by a circular area with a diameter of 6 millimeters (mm). The center point of the ETDRS grid may be the center of the circle. The central region may be a circle with a diameter of 1 mm. The ETDRS grid may be further divided into four inner and four outer regions by a circle concentric to the center with a diameter of 3 mm. The inner and outer regions may be each divided by four radial lines extending from the center circle to the outermost circle at, for example, 45°, 135°, 225°, and 315° and transecting the 3 mm circle in four places.

In one or more embodiments, a value for a feature (e.g., a number of fluid pockets, a volume of fluid, etc.) may be generated with respect to the macular center, the inner macular ring, or the outer macular ring. A value for a feature may be generated with respect to a particular region (e.g., quadrant) of the inner macular ring or outer macular ring. In some cases, a value for a feature may be generated with respect to two corresponding regions of the two rings (e.g., the superior inner region of the inner macular ring and the superior outer region of the outer macular ring). Thus, a value for a feature may be generated for any single region of the ETDRS grid, for a ring of the ETDRS grid, for a multi-region area formed by multiple regions of the ETDRS grid, or the central region of the ETDRS grid. More accurate retinal segmentation, as provided by the embodiments described herein, allows more accurate extraction of feature data with respect to the various regions and multi-region areas of the ETDRS grid.

In certain embodiments, once the image data 102 and/or the preprocessed image 116 are segmented and annotated to identify the layer element data 316 and the pathological element data 324, the feature extraction system 304 may determine one or more volumetric measurements (e.g., volume, thickness, area, extent, area of disruption, presence or absence of one or more disease-associated features, a number of disease-associated features) of the layer element data 316 and the pathological element data 324. In one or more embodiments, because the image data 102 and/or the preprocessed image 116 and the one or more 2D images (e.g., en face image, infrared image, thickness map) may be each known to correspond to the patient's macula, the one or more volumetric measurements determined from the image data 102 and/or the preprocessed image 116 may be then suitably mapped to the one or more 2D images (e.g., en face image, infrared image, thickness map) in accordance with ETDRS mapping and grid information 326.

In one or more embodiments, the feature extraction system 304 may utilize one or more image processing techniques (e.g., morphological image processing) to estimate the one or more volumetric measurements based on the ETDRS mapping and grid information 326, the known measurements and dimensions (e.g., three concentric circles with diameters of 1 mm, 3 mm, and 6 mm, respectively, and the four radial lines extending from the center circle to the outermost circle and transecting the 3 mm circle in four places), and the image data 102 and/or the preprocessed image 116 with respect to depth information (e.g., retinal layer depth). For example, in some embodiments, the feature extraction system 304 may derive a number of subspace constraints (e.g., an indication of the 3D subspaces, such as intraretinal subspaces and subretinal subspaces in which the DME-associated features may occur) based on the layer element data 316, and the derived number of subspace constraints may be then mapped and/or masked with respect to the nine subfields of the ETDRS grid.

In one or more embodiments, the feature extraction system 304 may then determine one or more volumetric measurements (e.g., volume, thickness, area, extent, area of disruption, presence, or absence of one or more disease-associated features, a number of one or more disease-associated features) of one or one more the layer element data 316 and/or the pathological element data 324. For example, the feature extraction system 304 may count the numbers of pixels per subfield of the ETDRS grid and convert the determined numbers of pixels per subfield of the ETDRS grid to microns for thicknesses, squared microns for areas, and cubic microns for volumes, and so forth. In one or more embodiments, one or more volumetric measurements may include a total volume of one or more retinal pathological elements, a fluid volume for one or more fluid pockets, or a thickness of one or more layers of the retina.

In one or more embodiments, the neural network system 310 is trained using training data 328. For example, the first set of neural networks 312 may be trained using a first training dataset of training data 328, while the second set of neural networks 314 may be trained using a second training dataset of the training data 328. The first training dataset may include, for example, without limitation, a plurality of training OCT images and training layer element data (e.g., a plurality of training layer element images). The second training dataset may include a plurality of training OCT images (which may be the same as, partially the same as, or different from the plurality of training OCT images in the first training dataset) and training pathological element data (e.g., a plurality of training pathological element images).

In some embodiments, the image data 102 and/or the preprocessed image 116 may additionally include one or more color fundus (CF) images, one or more fundus autofluorescence (FAF) images, one or more fluorescein angiography (FA) images, one or more other types of OCT images (e.g., OCT-A images), one or more other types of retinal images, or a combination thereof. In this manner, the image data 102 and/or the preprocessed image 116 may include multi-modal image input. Using multi-modal image input may increase the accuracy of the retinal segmentation.

In some embodiments, the retinal segmentation and feature extraction system 112 automatically performs segmentation of retinal images with better accuracy and consistency than expert human graders. The retinal segmentation and feature extraction system 112 provides a technical benefit of improving accuracy, reducing the overall computing resources, and/or reducing the time needed to perform segmentation of retinal images of subjects.

Further, using the retinal segmentation and feature extraction system 112 provides a technical improvement to the field of DME treatment and/or the technical field of segmenting retinal images of a subject. As noted above, the retinal segmentation and feature extraction system 112 automatically segments OCT images of a retina of a subject with better accuracy and consistency than expert human graders and may reduce the overall computing resources and/or time needed to segment images of subjects.

### IV. Example Methodologies for Use of the Retinal Segmentation and Feature Extraction System

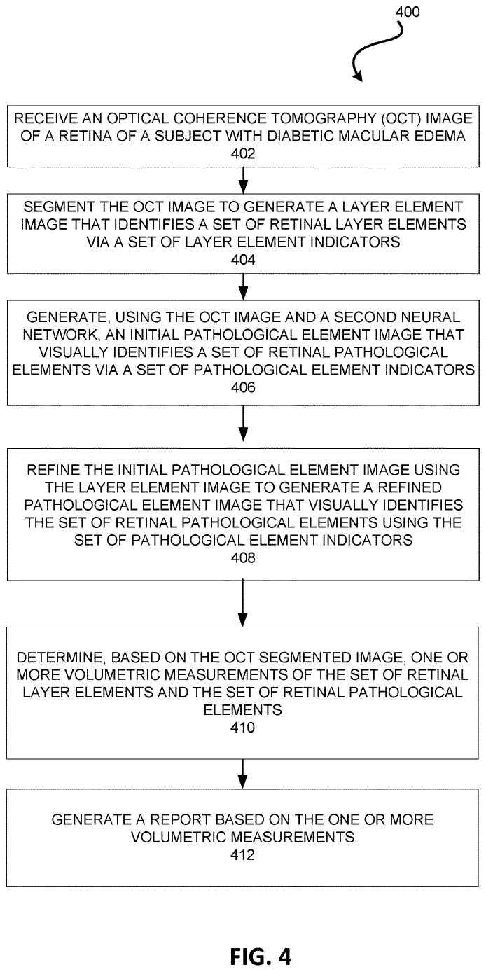

FIG. 4 is a flowchart of a process 400 of performing retinal segmentation and feature extraction, in accordance with various embodiments. In various embodiments, the method 400 can be implemented using the retinal segmentation and feature extraction system 112 as described in FIGS. 1 and 3.

As illustrated in FIG. 4, the process 400 includes, at step 402, receiving an optical coherence tomography (OCT) image of a retina of a subject with diabetic macular edema. In various embodiments, the OCT image may be the first OCT image 106 and/or one of the preprocessed images 116 in FIG. 1.

The process 400 further includes, at step 404, segmenting the OCT image to generate a layer element image that identifies a set of retinal layer elements via a set of layer element indicators, as discussed with respect to the layer element segmentation module 306 in FIG. 3. In some embodiments, the set of layer element indicators used in layer element image may be a set of graphical indicators as discussed above with respect to the layer element segmentation module 306 in FIG. 3. In one or more embodiments, the layer element image visually identifies one or more portions of the layer element image that have been identified as representing a retinal layer element of interest. The retinal layer element of interest may be, for example, a boundary associated with a retinal layer. In one or more embodiments, the layer element image may visually identify this retinal layer of interest by assigning a group of pixels that represent the boundary to a color that has been assigned to that retina boundary. When each retinal layer element of interest in the layer element image is a boundary, the layer element image may be referred to as an elevation map. Step 404 may be performed using a first neural network, such as first set of neural networks 312 in FIG. 3. The first neural network may include, for example, without limitation, at least one of a CNN, an FCN, a stacked FCN, a stacked FCN with multi-channel learning, a U-Net, or another type of neural network. In one or more embodiments, the first neural network is used to perform all the operations involved in step 404. In other embodiments, the first neural network is used to perform a portion of the operations involved in step 404.

The process 400 further includes, at step 406, generating an initial pathological element image that identifies a set of retinal pathological elements via a set of pathological element indicators. In some embodiments, the pathological element indicators assign a different group of pixels to each retinal pathological element of the set of retinal pathological elements. This identification may be an approximation. In one or more embodiments, the initial pathological element image visually identifies one or more portions of the initial pathological element image that have been identified as representing a retinal pathological element of interest. The retinal pathological element of interest may be, for example, intraretinal fluid. In one or more embodiments, the initial pathological element image visually identifies the intraretinal fluid by assigning a group of pixels that represent the intraretinal fluid to a color that has been assigned to the intraretinal fluid. Step 406 may be performed using a second neural network, such as the second set of neural networks 314 in FIG. 3. The second neural network may include, for example, without limitation, at least one of a CNN, an FCN, a stacked FCN, a stacked FCN with multi-channel learning, a U-Net, or another type of neural network. In one or more embodiments, the second neural network is used to perform all the operations involved in step 406. In other embodiments, the second neural network is used to perform a portion of the operations involved in step 406.

The process 400 further includes, at step 408 refining the initial pathological element image using the layer element image to generate a refined pathological element image that visually identifies the set of retinal pathological elements using the set of pathological element indicators. In some embodiments, the set of pathological element indicators assign an updated group of pixels to at least one retinal pathological element of the set of retinal pathological elements. The refined pathological element image more accurately represents at least one retinal pathological element of the set of retinal pathological elements as compared to the initial pathological element image.

The refining in step 408 may be performed in different ways. For example, the refining in step 408 includes updating a group of pixels in the initial pathological element image that is assigned to a particular retinal pathological element to form the updated group of pixels for the retinal pathological element in the refined pathological element image by constraining an allowable area for the retinal pathological element based on the layer element image. The allowable area may be constrained based on what is anatomically feasible, clinically relevant, and/or otherwise proper. The updated group of pixels includes fewer pixels than the group of pixels.

In one or more embodiments, a pathological element indicator of the set of pathological element indicators may be used to assign a group of pixels in the initial pathological element image to a first retinal pathological element of the set of retinal pathological elements. Refining the initial pathological element image may include reassigning a portion of the group of pixels in the initial pathological element image based on whether an anatomical characterization of the first retinal pathological element as identified by the pathological element indicator is anatomically feasible. The anatomical characterization of the first retinal pathological element of the set of retinal pathological elements may include at least one of a location, a size, a shape, a length, a width, a thickness, a volume of the retinal pathological element, or another characteristic.

In one or embodiments, the reassigning of the portion of the group of pixels may include, for example, reassigning a first pixel of the group of pixels from the first retinal pathological element to a second retinal pathological element of the set of retinal pathological elements based on the layer element image. Reassigning a pixel to a different retinal pathological element may include, for example, without limitation, changing the application of a pathological element indicator associated with that pixel. For example, the pixel may be changed from a first color in the initial pathological element image to a second color in the refined pathological element image.

In one or embodiments, the reassigning of the portion of the group of pixels may include, for example, reassigning a second pixel of the group of pixels from the first retinal pathological element to a background based on the layer element image. Reassigning a pixel to background may include, for example, without limitation, removing the application of a pathological element indicator associated with that pixel. For example, a color that was previously applied to that pixel in the initial pathological element image may be removed in the refined pathological element image.

The above examples of reassigning pixels are merely illustrative and are not meant to pose any limitations to the manner in which pixels may be reassigned. The reassigning of pixels in step 408 may be performed based on whether the anatomical characterization of the set of retinal pathological elements as presented by the set of pathological element indicators in initial pathological element image is allowable (e.g., anatomically feasible, clinically relevant, and/or otherwise proper). For example, a pixel annotated with a particular pathological element indicator that assigns that pixel to a particular retinal pathological element may be reassigned if the location of that pixel makes it anatomically infeasible to be associated with the particular retinal pathological element. Such determinations are made using the layer element image and/or data extracted from the layer element image.

In some embodiments, the image data 102 and/or the preprocessed image 116 being segmented and annotated to identify layer element data 316 and pathological element data 324 by the retinal segmentation system 302 results in the segmented image 118. As such, one output of the retinal segmentation system 302 is the segmented image(s) 118.

The process 400 further includes, at step 410, determining, based on the segmented OCT image (e.g., one of the segmented image(s) 118), one or more volumetric measurements of the set of retinal layer elements and the set of retinal pathological elements. In some embodiments, one or more volumetric measurements correspond to the ETDRS mapping and grid information. In various embodiments, the ETDRS mapping information and ETDRS grid information may be ETDRS mapping information and grid information 326 as described in FIG. 3. Step 408 may be performed, for example, by feature extraction system 304. As described with respect to FIG. 3, feature extraction system 304 may utilize one or more image processing techniques (e.g., morphological image processing) to determine a number of volumetric measurements based on the ETDRS mapping and grid information 326, the known measurements and dimensions (e.g., three concentric circles with diameters of 1 mm, 3 mm, and 6 mm, respectively, and the four radial lines extending from the center circle to the outermost circle and transecting the 3 mm circle in four places) of the ETDRS grid, and the image data 102 with respect to depth information (e.g., retinal layer depth).

In one or more embodiments, feature extraction system 304 may derive a number of region constraints (e.g., an indication of the 3D regions, such as intraretinal regions and subretinal regions in which the DME-associated features may occur) based on the set of retinal layer elements, and the derived number of region constraints may be then mapped and/or masked with respect to the nine regions of the ETDRS grid. Feature extraction system 304 may then determine one or more volumetric measurements (e.g., volume, thickness, area, extent, area of disruption, presence or absence of one or more DME-associated features, a number of one or more DME-associated features) by, for example, counting the numbers of pixels per region of the ETDRS grid and converting the determined numbers of pixels per region of the ETDRS grid to microns for thicknesses, squared microns for areas, and cubic microns for volumes, and so forth.

In one or more embodiments, the number of volumetric measurements may include, for example, a total volume of the one or more the DME-associated features (e.g., a total volume in cubic microns between various layers of the retina) with respect to one or more of the nine regions of the ETDRS grid, a fluid volume of the one or more fluid features (e.g., a total volume in cubic microns for various fluids) with respect to one or more of the nine regions of the ETDRS grid, a deposit volume of the one or more deposit features (e.g., a total volume in cubic microns for various deposit materials) with respect to one or more of the nine regions of the ETDRS grid, a thickness of one or more of the layers of the retina (e.g., thickness measurements in microns of all of the layers of the retina, thickness measurements in microns with respect to one or more specific layers of the retina, or thickness measurements in microns of the “slabs” or spaces between the layers of the retina) with respect to one or more of the nine regions of the ETDRS grid, a fluid extent of the one or more fluid features (e.g., volume measurements in microns for various fluids) with respect to one or more of the nine regions of the ETDRS grid, or a number of the one or more deposit features (e.g., a numerical value representing the total number of identified deposit materials) with respect to one or more of the nine regions of the ETDRS grid.

In certain embodiments, the number of volumetric measurements may further include, for example, an area of the one or more DME-associated features (e.g., en face image area measurements in squared microns for various fluids or deposit materials) with respect to one or more of the nine regions of the ETDRS grid, an indication of a presence or an absence of the one or more DME-associated features (e.g., a binary value representing either a presence or an absence of various fluids and deposit materials) with respect to one or more of the nine regions of the ETDRS grid, or an area of disruption of the one or more DME-associated features (e.g., en face image area of disruption in squared microns for various fluids or deposit materials) with respect to one or more of the nine regions of the ETDRS grid.

The process 400 further includes, at step 412 generating a report based on the one or more volumetric measurements. In one or more embodiments, a clinical report may include, for example, a table, a chart, an XML file, a HTML file, a spreadsheet, a text file, an image file, a graphics file, a hyperlink, a webpage, or other file that may be accessible and viewable on a computing device by the one or more the clinicians (e.g., ophthalmologists, optometrists, or other retinal specialists). In one or more embodiments, the report only includes the one or more volumetric measurements. In some embodiments, the report may include additional information such as patient treatment information and patient best corrected visual acuity scores (BCVA). In one or more embodiments, the report is displayed on display system 130 in FIG. 1. In some embodiments, the step 412 includes generating the retinal data 120 by extracting feature data from the refined pathological element image and in some cases, from the layer element image.

In various embodiments, the first neural network described in step 404, the second neural network described in step 406, or both may be trained using training data such as training data 328 in FIG. 3. The first neural network may be trained using, for example, a first training dataset comprising a first plurality of training OCT images and a plurality of training layer element images. The plurality of training layer element images may include training multi-channel maps, training initial layer element images, training refined layer element images, or a combination thereof.

In some embodiments, the method 400 generates a refined pathological element image (e.g., one of the segmented image(s) 118) with better accuracy and consistency than human graders. The method 400 provides a technical effect of improving accuracy, reducing the overall computing resources, and/or reducing the time needed to provide a refined pathological element image identifying both retinal layer elements and retinal pathological elements.

In some embodiments, the method 400 provides a technical improvement to the field of automated segmentation of OCT images and/or the technical field of predicting DME treatment response. As noted above, method 400 generates a refined pathological element image with better accuracy and consistency than human graders and may reduce the overall computing resources and/or time needed to automatically segment OCT images of subjects. In some embodiments, the method 400 includes a new combination of steps that results in the technical improvement over conventional segmentation methods.

### V. Example Implementation for Predicting Treatment Response in DME

An example experiment associated with predicting future treatment response in patients with DME was conducted. This example experiment was conducted using a segmentation model, such as the retinal segmentation and feature extraction system 112 in FIG. 1. This example experiment may be conducted by process 300 described above.

### V.A. Example Methodology of Training Models

One or more models described above, such as, for example, neural network system 310 in FIG. 3, may be trained in different ways. In one or more embodiments, the model is trained with a training dataset (e.g., training data 328 in FIG. 3) that includes one or more training OCT volume images. Each of these training OCT volume images may be of a different retina that has been identified as having DME. One or more models described above may be trained using a first data set and a first data strategy to create a trained model to automatically segment OCT images to predict treatment response for subjects with DME.

In an example implementation, a deep learning neural network, for example neural network system 310 in FIG. 3, was trained using a first data set. For example, the first data set may be training data 328 in FIG. 1. In one or more embodiments, the first data set included OCT B-Scans from Phase 2 BOULEVARD (NCTR02699450) trials at baseline, week 4, and week 52. In one or more embodiments, the first data set included measurements for intraretinal fluid (IRF) and subretinal fluid (SRF) volume, and total retinal, outer nuclear layer (ONL), and inner retinal thickness averaged over the entire 3-mm diameter of an ETDRS region. In one or more embodiments, the first data strategy included pooling the patients of the first data set and splitting the patients so that 10% of the first dataset patient data was held out for validation.

### V.B. Example Methods for Automated Retinal Segmentation

After training the segmentation model, the trained segmentation model was used to segment the OCT images and generate layer element data and pathological element data. The trained segmentation model may be, for example the retinal segmentation system 302 in FIG. 3. In an example implementation, image data, such as image data 102 in FIG. 1, included OCT images, such as first OCT image 106 in FIG. 1. Image input data included images of eyes for 887 patients diagnosed with DME from Phase 3 YOSEMITE (NCT03622580) and RHINE (NCT03622593) trials. Treatment information was analyzed for each patient and included an indication of whether the patient received treatment with Faricimab or aflibercept. Clinical data including best correct visual acuity (BCVA) scores at baseline, week 4, and week 52 were compiled.

The layer element data may be, for example, layer element data 316 in FIG. 3. The pathological element data may be, for example, pathological element data 324 in FIG. 3. The generation of layer element data and retinal element data may be conducted, for example, by steps 404, 406, 408, and/or 410 in process 400 (FIG. 4).

In this example implementation, the OCT images were associated with ETDRS mapping and grid information, such as ETDRS mapping and grid information 326 in FIG. 3. The ETDRS mapping and grid information identified the location of one or more features of interest or areas of interest in terms of the nine regions of the ETDRS grid.

In this example implementation, measurements were generated for IRF and SRF volume, and total retinal, ONL, and inner retinal thickness based on the OCT images. This may be conducted, for example, by the feature extraction system 304. This may be conducted, for example, by step 410 in process 400 (FIG. 4). The measurements were generated for IRF and SRF volume, and total retinal, ONL, and inner retinal thickness at baseline (before treatment) and weeks 4 and 52 after patients began receiving treatment (i.e., with Faricimab or aflibercept). The measurements were averaged over the 3-mm diameter of the EDTRS region.

In this example implementation, after the measurements were generated for the OCT images, patients were separated into three groups based on the measured IRF volume reduction between baseline and week 4. The three groups consisted of patients that had less than 20% IRF volume reduction, 20-50% IRF volume reduction, or greater than 50% IRF volume reduction between baseline and week 4. In some embodiments, this grouping may be conducted manually by human analysts. In other embodiments, this grouping may be conducted by a system, such as computing platform 108 in FIG. 1.

Further, in this example implementation, retinal fluid volumes, retinal thicknesses, and BCVA changes between the baseline measurements and the week 4 measurements were compared. In some embodiments, the comparison may be conducted manually by human analysts. In other embodiments, this comparison may be conducted by a system, such as computing platform 108 in FIG. 1.

### V.C. Example Results

In this example, after the OCT images were segmented by the example model and the measurements were generated for the retinal layer data and the pathological element data of interest identified above (i.e., IRF and SRF volume, and total retinal, ONL, and inner retinal thickness), a report was generated. This step may be conducted, for example, by step 412 in process 400 (FIG. 4). As another example, this step may be generated by a computing system, such as computing platform 108 in FIG. 1.

In this example implementation, the report showed at week 4, IRF volume was reduced by less than 20% in 242 patients, 20-50% in 248 patients, and greater than 50% in 397 patients. For the patients that experienced less than 20% IRF volume reduction at week 4, at week 52 this group of patients experienced IRF volume reduction of 270 nL, total retinal thickness reduction of 87 um, ONL thickness reduction of 48 um, and inner retinal thickness decreased by 34 um. For the patients that experienced 20-50% IRF volume reduction at week 4, at week 52 this group of patients experienced IRF volume reduction of 378 nL, total retinal thickness reduction of 110 um, ONL thickness reduction of 57 um, and inner retinal thickness decreased by 46 um. For the patients that experienced greater than 50% IRF volume reduction at week 4, at week 52 this group of patients experienced IRF volume reduction of 410 nL, total retinal thickness reduction of 122 um, ONL thickness reduction of 60 um, and inner retinal thickness decreased by 55 um. In this example implementation, there was no difference in SRF volume reduction from baseline between groups at week 52.

In this example implementation, the report additionally showed that for the patients that experienced less than 20% IRF volume reduction at week 4, at week 52 this group of patients had BCVA scores of 8.2 letters. For the patients that experienced 20-50% IRF volume reduction at week 4, at week 52 this group of patients had BCVA scores of 10.6 letters. For the patients that experienced greater than 50% IRF volume reduction at week 4, at week 52 this group of patients had BCVA scores of 12.6 letters. In this example implementation, there was no difference in the SRF volume reduction from baseline and between groups at week 52.

V.D. Example Conclusions

In this example implementation, the generated report showed that patients with greater than 50% IRF volume reduction at week 4 showed greater improvements in their BCVA scores than patients in the other two groups. This suggests that greater IRF volume reduction within 1 month of starting treatment improved one-year anatomical and visual outcomes in patients diagnosed with DME. This finding may be used to better predict treatment outcomes in patients with DM and develop treatment plans accordingly.

In various embodiments, whether a subject with DME experiences greater than 50% IRF volume reduction within 4 weeks of starting treatment may also be used to determine whether a subject is a candidate for a clinical trial, to which clinical trial to assign a subject, how to customize a treatment for a subject, how to monitor progress of the subject during a clinical trial, or a combination thereof. In various embodiments, this exemplary conclusion may be used to enroll the subject in a clinical trial, exclude the subject from participating in a clinical trial, customize a protocol in a clinical trial for a subject, or enroll the subject in a different clinical trial.

In various embodiments, the exemplary conclusion may also be used to develop, implement, or change a treatment plan for a subject with DME. For example, if the subject with DME experiences greater than 50% reduction in IRF volume within 4 weeks of starting treatment, it may be an indication that the treatment regime for the subject is effective in treating the patient's DME symptoms or progression. Thus, one or more clinicians (e.g., ophthalmologists, optometrists, or other retinal specialists) may decide, based on the volumetric measurements produced by the feature extraction system, that the current treatment regime the patient is on should be continued.

In other embodiments, if the subject with DME experiences less than 50% reduction in IRF volume within 4 weeks of starting treatment, it may be an indication that the treatment regime for the patient is not as effective at treating that patient's DME symptoms or progression. Thus, one or more clinicians (e.g., ophthalmologists, optometrists, or other retinal specialists) may decide that a different treatment regime should be developed for the patient. This could include, for example, a different medication, a different injection frequency, or admission into a different clinical trial.

### VI. Computer-Implemented System

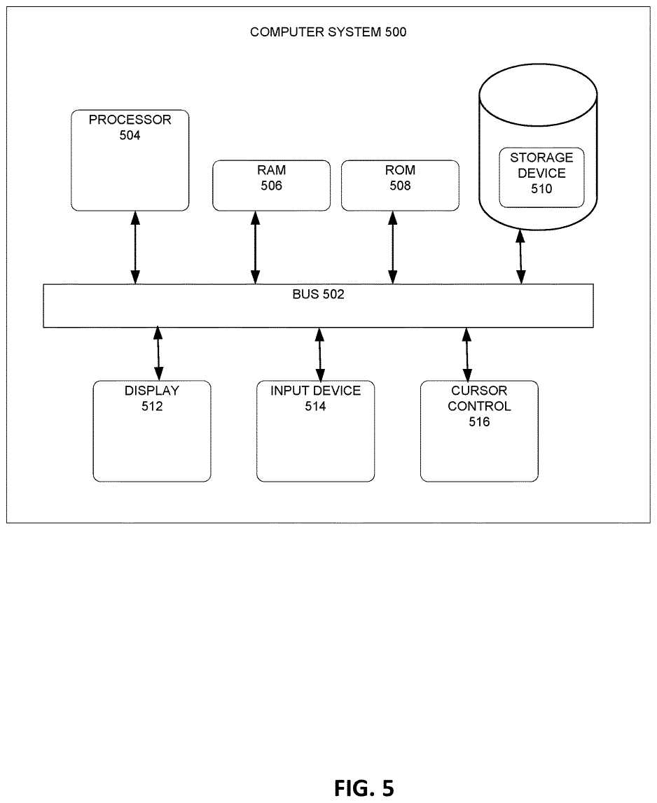

FIG. 5 is a block diagram that illustrates a computer system, in accordance with various embodiments. Computer system 500 may be one example of an implementation for computing platform 108 in FIG. 1. In various embodiments of the present teachings, computer system 500 can include a bus 502 or other communication mechanism for communicating information, and a processor 504 coupled with bus 502 for processing information. In various embodiments, computer system 500 can also include a memory, which can be a random access memory (RAM) 506 or other dynamic storage device, coupled to bus 502 for determining instructions to be executed by processor 504. Memory also can be used for storing temporary variables or other intermediate information during execution of instructions to be executed by processor 504. In various embodiments, computer system 500 can further include a read only memory (ROM) 508 or other static storage device coupled to bus 502 for storing static information and instructions for processor 504. A storage device 510, such as a magnetic disk or optical disk, can be provided and coupled to bus 502 for storing information and instructions.

In various embodiments, computer system 500 can be coupled via bus 502 to a display 512, such as a cathode ray tube (CRT) or liquid crystal display (LCD), for displaying information to a computer user. An input device 514, including alphanumeric and other keys, can be coupled to bus 502 for communicating information and command selections to processor 504. Another type of user input device is a cursor control 516, such as a mouse, a trackball or cursor direction keys for communicating direction information and command selections to processor 504 and for controlling cursor movement on display 512. This input device 514 typically has two degrees of freedom in two axes, a first axis (i.e., W) and a second axis (i.e., y), that allows the device to specify positions in a plane. However, it should be understood that input devices 514 allowing for 5-dimensional (x, y and 5) cursor movement are also contemplated herein.

Consistent with certain implementations of the present teachings, results can be provided by computer system 500 in response to processor 504 executing one or more sequences of one or more instructions contained in memory 506. Such instructions can be read into memory 506 from another computer-readable medium or computer-readable storage medium, such as storage device 510. Execution of the sequences of instructions contained in memory 506 can cause processor 504 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 504 for execution. Such a medium can take many forms, including but not limited to, non-volatile media, volatile media, and transmission media. Examples of non-volatile media can include, but are not limited to, optical, solid state, magnetic disks, such as storage device 510. Examples of volatile media can include, but are not limited to, dynamic memory, such as memory 506. Examples of transmission media can include, but are not limited to, coaxial cables, copper wire, and fiber optics, including the wires that comprise bus 502.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 504 of computer system 500 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 500 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 500, whereby processor 504 would execute the analyses and determinations provided by these engines, subject to instructions provided by any one of, or a combination of, memory components RAM 506, ROM 508, and/or storage device 510 and user input provided via input device 514.

### VII. Example Definitions and Context

The disclosure is not limited to the example embodiments and applications described herein or to the manner in which the example embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “included in” can be “connected to”, “attached to”, “associated with”, or “mapped to.”

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 1, 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be used. The item may be a particular object, thing, step, operation, process, or category. In other words, “at least one of” means any combination of items or number of items may be used from the list, but not all of the items in the list may be used. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may include the practice of using algorithms to parse data, learn from the data, and then make a determination or prediction about something in the world. Machine learning may use algorithms that can learn from data without relying on rules-based programming. Deep learning may be one form of machine learning.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks may include one or more hidden layers in addition to an output layer. The output of each hidden layer may be used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), a U-Net, a fully convolutional network (FCN), a stacked FCN, a stacked FCN with multi-channel learning, a Squeeze and Excitation embedded neural network, a MobileNet, or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### VIII. Recitation of Example Embodiments

Embodiment 1: A method comprising: receiving optical coherence tomography (OCT) imaging data for a retina of a subject with diabetic macular edema (DME), wherein the OCT imaging data comprises: a first OCT image associated with a first point in time and a second OCT image associated with a second point in time that is after the first point in time; generating, via a machine learning model, a first OCT segmented image using the first OCT image and a second OCT segmented image using the second OCT image; generating, based on the first OCT segmented image, a first measurement of a DME-associated feature; generating, based on the second OCT segmented image, a second measurement of the DME-associated feature; identifying a reduction between the first and second measurement; comparing the reduction to a threshold; wherein the reduction exceeding the threshold is associated with an improved vision health metric for the subject at a third point in time that is after the second point in time; and generating a treatment output based on the comparison.

Embodiment 2: The method of embodiment 1, wherein the first point in time is a baseline point in time before a first DME treatment protocol has been administered to the subject; wherein the second point in time is between about 2 weeks and about 6 weeks after the first DME treatment has been administered to the subject; and wherein the third point in time is between about 46 and about 58 weeks after the first DME treatment has been administered to the subject.

Embodiment 3: The method of any one of embodiments 1-2, wherein each of the first measurement and the second measurement is a volumetric measurement of the DME-associated feature.

Embodiment 4: The method of any one of embodiments 1-3, wherein the DME-associated feature is intraretinal fluid (IRF).

Embodiment 5: The method of embodiment 4, wherein the threshold is a 50% reduction of the intraretinal fluid (IRF) volume for the subject between the first point in time and the second point in time.

Embodiment 6: The method of any one of embodiments 2-5, wherein the first DME treatment protocol is a monoclonal antibody treatment.

Embodiment 7: The method of any one of embodiments 2-5, wherein the first DME treatment protocol is an anti-Vascular Endothelial Growth Factor (anti-VEGF) treatment.

Embodiment 8: The method of any one of embodiments 2-7, wherein the treatment output identifies that the first DME treatment protocol for the subject should be continued when the reduction between the first and second measurement is greater than the threshold.

Embodiment 9: The method of any one of embodiments 2-8, wherein the treatment output is identifying that a second DME treatment protocol should be developed for the subject when the reduction between the first and second measurement is lower than the threshold.

Embodiment 10: The method of any one of embodiments 1-9, wherein the improved vision health metric comprises an improved visual acuity.

Embodiment 11: The method of any one of embodiments 1-10, wherein the improved vision health metric is relative to a vision health metric of the subject when the reduction does not exceed the threshold.

Embodiment 12: The method of any one of embodiments 1-11, wherein each of the first measurement and the second measurement is a volumetric measurement of the DME-associated feature; and wherein generating the volumetric measurement of the DME-associated feature comprises determining a total volume, a fluid volume, a deposit volume, an area, or a thickness of the DME-associated feature

Embodiment 13: The method of any one of embodiments 1-12, further comprising generating a report comprising the reduction and the treatment output

Embodiment 14: The method of embodiment 13, further comprising analyzing the report for use in detection, diagnosis, and treatment of the subject.

Embodiment 15: The method of any one of embodiments 1-14, wherein the machine learning model includes a deep learning model.

Embodiment 16: The method of any one of embodiments 1-15, further comprising forming, using the first OCT image, a first image input for the machine learning model; and wherein generating, via the machine learning model, the first OCT segmented image using the first OCT image comprises generating, via the machine learning model, the first OCT segmented image using the first image input.

Embodiment 17: The method of embodiment 16, further comprising forming, using the second OCT image, a second image input for the machine learning model; and wherein generating, via the machine learning model, the second OCT segmented image using the second OCT image comprises generating, via the machine learning model, the second OCT segmented image using the second image input.

Embodiment 18: The method of any one of embodiments 1-17, wherein each of the first OCT segmented image and the second OCT segmented image identifies one or more layer features corresponding to layers of the retina and one or more DME-associated features associated with the one or more layer features.

Embodiment 19: A system comprising: one or more data processors; and a non-transitory computer readable storage medium containing instructions which, when executed on the one or more data processors, cause the one or more data processors to perform part or all of one or more methods disclosed in embodiments 1-18.

Embodiment 20: A computer-program product tangibly embodied in a non-transitory machine-readable storage medium, including instructions configured to cause one or more data processors to perform part or all of one or more methods disclosed in embodiments 1-18.

### IX. Additional Considerations

The headers and subheaders between sections and subsections of this document are included solely for improving readability and do not imply that features cannot be combined across sections and subsection. Accordingly, sections and subsections do not describe separate embodiments. Any one or more of the embodiments described herein in any section or with respect to any FIG. may be combined with or otherwise integrated with any one or more of the other embodiments described herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure here a computer-program product tangibly embodied in a non-transitory machine-readable storage medium, including instructions configured to cause one or more data processors to perform part or all of one or more methods and/or part or all of one or more processes disclosed herein.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

For example, the flowcharts and block diagrams described above illustrate the architecture, functionality, and/or operation of possible implementations of various method and system embodiments. Each block in the flowcharts or block diagrams may represent a module, a segment, a function, a portion of an operation or step, or a combination thereof. In some alternative implementations of an embodiment, the function or functions noted in the blocks may occur out of the order noted in the figures. For example, in some cases, two blocks shown in succession may be executed substantially concurrently. In other cases, the blocks may be performed in the reverse order. Further, in some cases, one or more blocks may be added to replace or supplement one or more other blocks in a flowchart or block diagram.

Thus, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

1. A method, comprising:
receiving optical coherence tomography (OCT) imaging data for a retina of a subject with diabetic macular edema (DME), wherein the OCT imaging data comprises:
a first OCT image associated with a first point in time; and
a second OCT image associated with a second point in time that is after the first point in time;
generating, via a machine learning model, a first OCT segmented image using the first OCT image and a second OCT segmented image using the second OCT image;
generating, based on the first OCT segmented image, a first measurement of a DME-associated feature;
generating, based on the second OCT segmented image, a second measurement of the DME-associated feature;
identifying a reduction between the first and second measurement;
comparing the reduction to a threshold;
wherein the reduction exceeding the threshold is associated with an improved vision health metric for the subject at a third point in time that is after the second point in time;
and
generating a treatment output based on the comparison.

2. The method of claim 1,
wherein the first point in time is a baseline point in time before a first DME treatment protocol has been administered to the subject;
wherein the second point in time is between about 2 weeks and about 6 weeks after the first DME treatment has been administered to the subject; and
wherein the third point in time is between about 46 and about 58 weeks after the first DME treatment has been administered to the subject.

3. The method of claim 1, wherein each of the first measurement and the second measurement is a volumetric measurement of the DME-associated feature.

4. The method of claim 1, wherein the DME-associated feature is intraretinal fluid (IRF).

5. The method of claim 4, wherein the threshold is a 50% reduction of the intraretinal fluid (IRF) volume for the subject between the first point in time and the second point in time.

6. The method of claim 2, wherein the first DME treatment protocol is a monoclonal antibody treatment.

7. The method of claim 2, wherein the first DME treatment protocol is an anti-Vascular Endothelial Growth Factor (anti-VEGF) treatment.

8. The method of claim 2, wherein the treatment output identifies that the first DME treatment protocol for the subject should be continued when the reduction between the first and second measurement is greater than the threshold.

9. The method of claim 2, wherein the treatment output is identifying that a second DME treatment protocol should be developed for the subject when the reduction between the first and second measurement is lower than the threshold.

10. The method of claim 1, wherein the improved vision health metric comprises an improved visual acuity.

11. The method of claim 1, wherein the improved vision health metric is relative to a vision health metric of the subject when the reduction does not exceed the threshold.

12. The method of claim 1,
wherein each of the first measurement and the second measurement is a volumetric measurement of the DME-associated feature; and
wherein generating the volumetric measurement of the DME-associated feature comprises determining a total volume, a fluid volume, a deposit volume, an area, or a thickness of the DME-associated feature.

13. The method of claim 1, further comprising generating a report comprising the reduction and the treatment output.

14. The method of claim 13, further comprising analyzing the report for use in detection, diagnosis, and treatment of the subject.

15. The method of claim 1, wherein the machine learning model includes a deep learning model.

16. The method of claim 1, further comprising forming, using the first OCT image, a first image input for the machine learning model; and
wherein generating, via the machine learning model, the first OCT segmented image using the first OCT image comprises generating, via the machine learning model, the first OCT segmented image using the first image input.

17. The method of claim 16, further comprising forming, using the second OCT image, a second image input for the machine learning model; and
wherein generating, via the machine learning model, the second OCT segmented image using the second OCT image comprises generating, via the machine learning model, the second OCT segmented image using the second image input.

18. The method of claim 1, wherein each of the first OCT segmented image and the second OCT segmented image identifies one or more layer features corresponding to layers of the retina and one or more DME-associated features associated with the one or more layer features.

19. A system comprising:
one or more data processors; and
a non-transitory computer readable storage medium containing instructions which, when executed on the one or more data processors, cause the one or more data processors to:
receive optical coherence tomography (OCT) imaging data for a retina of a subject with diabetic macular edema (DME), wherein the OCT imaging data comprises:
a first OCT image associated with a first point in time; and
a second OCT image associated with a second point in time that is after the first point in time;
generate, via a machine learning model, a first OCT segmented image using the first OCT image and a second OCT segmented image using the second OCT image;
generate, based on the first OCT segmented image, a first measurement of a DME-associated feature;
generate, based on the second OCT segmented image, a second measurement of the DME-associated feature;
identify a reduction between the first and second measurement;
compare the reduction to a threshold;
wherein the reduction exceeding the threshold is associated with an improved vision health metric for the subject at a third point in time that is after the second point in time;
and
generate a treatment output based on the comparison.

20. A computer-program product tangibly embodied in a non-transitory machine-readable storage medium, including instructions configured to cause one or more data processors to:
receive optical coherence tomography (OCT) imaging data for a retina of a subject with diabetic macular edema (DME), wherein the OCT imaging data comprises:
a first OCT image associated with a first point in time; and
a second OCT image associated with a second point in time that is after the first point in time;
generate, via a machine learning model, a first OCT segmented image using the first OCT image and a second OCT segmented image using the second OCT image;
generate, based on the first OCT segmented image, a first measurement of a DME-associated feature;
generate, based on the second OCT segmented image, a second measurement of the DME-associated feature;
identify a reduction between the first and second measurement;
compare the reduction to a threshold;
wherein the reduction exceeding the threshold is associated with an improved vision health metric for the subject at a third point in time that is after the second point in time;
and
generate a treatment output based on the comparison.
